(19) United States
(12) Patent Application Publication
(10) Pub. No.: US 2015/0094818 A1
(43) Pub. Date: Apr. 2, 2015

(54) PROSTHESIS PART

(71) Applicant: WALDEMAR LINK GMBH & CO. KG, Hamburg (DE)

(72) Inventors: Marco Iredi, Norderstedt (DE); Melanie Stangel, Barmstedt (DE)

(73) Assignee: WALDEMAR LINK GMBH & CO. KG, Hamburg (DE)

(21) Appl. No.: 14/383,032

(22) PCT Filed: Mar. 4, 2013
(86) PCT No.: PCT/EP2013/054252

§ 371 Date: Oct. 24, 2014

(30) Foreign Application Priority Data
Mar. 9, 2012 (EP) 12158698.6

(51) Int. Cl. A61F 2/30

(52) U.S. Cl. CPC A61F 2/30721; A61F 2002/30205; A61F 2002/30405; A61F 2002/30332
USPC 623/18.11

(57) ABSTRACT

The invention relates to a prosthesis part having a conical protrusion for connecting to an additional prosthesis part having a complementary conical bore, wherein the conical protrusion has a lateral recess for the engagement of a securing element, and wherein the prosthesis part with the conical protrusion has a removable cone attachment, said cone attachment having a lateral recess for the engagement of a securing element. The invention further relates to a prosthesis having a prosthesis part according to the invention and a set of prosthesis parts, wherein a prosthesis part according to the invention is formed.

The invention relates to a prosthesis part and in particular to its configuration for connection to an additional prosthesis part.

It is known that a human being's bones and joints can be replaced by prostheses if damaged, for example as a result of illness or accidents. It is thus possible for a patient to regain the original functionality of his or her bone structure. Since corresponding prostheses generally remain permanently in the patient's body, one also talks of endoprostheses.

Corresponding prostheses are often made up of two or more individual parts which can be assembled as required. Thus, a prosthesis can be made up, for example, of a main prosthesis part and a prosthesis head. Such multi-part prostheses offer the advantage that relatively few interchangeable individual parts can be extensively combined with one another so as to thus create a prosthesis suitable for a specific patient. Accordingly, a prosthesis does not have to be produced specifically for one patient, but can also be adapted to the patient's circumstances in situ, i.e. even during the surgical intervention for the implantation of a prosthesis.

Moreover, due to the possibility of assembling a prosthesis from a number of prosthesis parts in situ, the surgical preparations for the implantation of a prosthesis can be less extreme. If in doubt, one only need remove the bone material which is to be replaced by the individual prosthesis parts. One can often avoid removing any additional bone in order to be able to pass parts of a prosthesis through bone regions in which ultimately no or other, possibly smaller parts of the prosthesis are positioned in the implanted state. In fact, the individual prosthesis parts can generally be implanted separately from one another and only be joined together subsequently. Since the surgeon in question has to implant a plurality of smaller, generally easily manageable prosthesis parts instead of a single prosthesis that may be difficult to handle, and only has to connect them subsequently, his work is made considerably easier.

In the prior art various possibilities are known for interconnecting individual prosthesis parts securely to form a single prosthesis. It is thus known, for example, for a prosthesis part to have a conical protrusion for connecting to an additional prosthesis part having a complementary conical bore. Furthermore, it is known for corresponding conical connections to be secured against undesirable loosening by locking screws. Here the locking screw is passed through a bore of the one prosthesis part and engages at least partially in an indentation or bore on the other prosthesis part.

It is the object of the present invention to improve conical connections of prosthesis parts and prostheses.

This object is achieved by a prosthesis part, a prosthesis and a set comprising at least three prosthesis parts according to claims 1, 7 and 10. Advantageous further developments emerge from the other dependent claims.

Accordingly, the invention relates to a prosthesis part having a conical protrusion for connecting to an additional prosthesis part having a complementary conical bore, the conical protrusion having a lateral recess for the engagement of a securing element and the prosthesis part having the conical protrusion having a removable cone attachment that has a lateral recess for the engagement of a securing element.

Furthermore, the invention relates to a prosthesis comprising a prosthesis part according to the invention having a conical protrusion, a prosthesis part having a complementary conical bore and a securing element, the prosthesis part having the conical bore having a through-hole through which the securing element is passed so that the securing element can be engaged with the lateral recess of the removable cone attachment of the prosthesis part having the conical protrusion inserted into the prosthesis part having the conical bore, the securing element being a headless screw the length of which is chosen so that, if the removable cone attachment or the prosthesis part having the conical protrusion is missing, it can fall out of the through-hole so that the securing element is passed into the interior of the prosthesis part having the conical bore.

Moreover, the invention relates to a set comprising a first prosthesis part according to the invention having a conical protrusion, a second and a third prosthesis part each having a complementary conical bore, the second prosthesis part having a through-hole through which a securing element is passed so that the securing element can be engaged with the lateral recess of the removable cone attachment of the first prosthesis part inserted into the second prosthesis part, and the third prosthesis part having a through-hole through which a securing element is passed so that the securing element can be engaged with the lateral recess of the conical protrusion of the first prosthesis part that can be inserted into the third prosthesis part.

Since the prosthesis part according to the invention also has, as a possibility for the engagement of a securing element, another lateral recess on a removable cone attachment in addition to a lateral recess on the conical protrusion, it can be combined extremely flexibly with additional prosthesis parts.

The prosthesis part according to the invention can thus, for example, be combined with a prosthesis part wherein the conical bore is made with a thin wall, i.e. the wall surrounding the bore is thin. Generally, with a conical bore made with a thin wall, a through-hole through which a securing element can be passed so as to engage with a lateral recess on a prosthesis part having a conical protrusion cannot be provided. Specifically, with a corresponding through-hole it would not be sufficiently guaranteed that it will not shear if tension is applied to the conical connection. For the case of a thin-walled, conical bore, with the prosthesis part according to the invention the securing element can also engage in the lateral recess on the cone attachment of the prosthesis part according to the invention. The securing element then no longer needs to be guided through thin-walled regions of the additional prosthesis part. In fact, the through-hole provided for this purpose can be guided through regions away from the conical bore which do not generally have thin walls. The securing of the conical connection can thus also be guaranteed for prosthesis parts having thin-walled, conical bores.

In a case where the prosthesis part according to the invention is to be connected to an additional prosthesis part in which the conical bore is made with a thick wall, a securing element guided through a through-hole on this additional prosthesis part can engage directly in the lateral recess on the conical protrusion. In this regard, “thick-walled” means that at the point of a through-hole through which a securing element is passed, the wall has a sufficient wall thickness so that it is not anticipated that the through-hole will shear when subjected to the maximum loads anticipated (optionally plus a safety factor).

Before the prosthesis part according to the invention and an additional prosthesis part having a thick-walled conical bore are connected to one another, the cone attachment can first of all be removed from the prosthesis part according to the invention. Since the cone attachment is designed to be removable, this is easily possible. The removal of the cone attachment makes it possible to also use the prosthesis part according to the invention with additional prosthesis parts in which no cavity is provided for receiving the cone attachment or a corresponding cavity cannot be created either due to the configuration of the additional prosthesis part.

The prosthesis part according to the invention can therefore be connected to a plurality of additional prosthesis part types. The prosthesis part according to the invention makes it possible here for securing by the engagement of a securing element in a lateral recess to be guaranteed independently of the configuration of the additional prosthesis part. Since the prosthesis part according to the invention enables various securings of a conical connection, it can be used extremely flexibly and can be combined with additional prosthesis parts.

The lateral recess for the engagement of a securing element on the conical protrusion of the prosthesis part according to the invention can preferably be aligned perpendicularly to the cone axis. The recess can, for example, be in the form of a circumferential indentation or groove, but also of a blind hole.

The cone attachment is preferably rotationally symmetrical, and more preferably cylindrical in form. The recess on the cone attachment is preferably aligned perpendicularly to its axis and can be in the form, for example, of a circumferential indentation or groove or in the form of a blind hole. It is preferred if the axis of symmetry of the cone attachment in the assembled state coincides with the axis of the conical protrusion.

Preferably, the cone attachment is connected to the conical protrusion such as to be removable by means of a screw connection. A screw connection offers the advantage of a secure connection with at the same time simple releasability or removability of the cone attachment from the conical protrusion. The cone attachment can have an extension in the form of a screw, and the conical protrusion can have a blind hole with an internal thread in order to form the screw connection.

Furthermore, the invention relates to a prosthesis which, in addition to the prosthesis part described above, also comprises an additional prosthesis part. This additional prosthesis part has a conical bore which is complementary to the conical protrusion of the first mentioned prosthesis part. In other words, the two prosthesis parts are designed to be connected by a conical connection.

In the additional prosthesis part a through-hole is provided through which a securing element is passed and which is arranged such that a securing element passed through here can basically engage in the lateral recess on the cone attachment of the other prosthesis part if the two prosthesis parts are connected to one another.

The securing element is preferably a headless screw. For guiding the headless screw, the through-hole in the prosthesis part having the conical bore can have at least partially an internal thread in which the external thread of the headless screw can engage.

It is preferred if the length of the headless screw is chosen such that, if the removable cone attachment on the prosthesis part having the conical protrusion is missing, it can fall out of the through-hole in order to pass the securing element into the interior of the prosthesis part having the conical bore. Here, the headless screw can fall into the conical bore of the corresponding prosthesis part or into another cavity.

If the two prosthesis parts of a prosthesis are assembled, i.e. the conical connection is established between the two prosthesis parts, it is no longer evident whether there is a cone attachment on the prosthesis part having the conical protrusion. A visual check is then no longer possible without releasing the conical connection again. Since the length of the headless screw is chosen as described, it can be ensured that, in the event of the cone attachment being missing, the surgeon is given a clear indication of this. In this case the securing element or the headless screw will not be able to be tightened, but in fact will fall into the conical bore of the additional prosthesis part or a comparable cavity. In this way it is indicated to the surgeon that the cone attachment is missing and that the cone connection between the two prosthesis parts is not secured.

In the prosthesis described above, the conical bore in the one prosthesis part can clearly be made with a thin wall. That is to say, the through-hole for passing through the securing element must not lead through the wall surrounding the conical bore, but rather can be disposed in a different region of the prosthesis part in which shearing of the through-hole is not anticipated. However, the advantages described also arise, needless to say, with a prosthesis having a conical bore in the additional prosthesis part made with a thick wall.

The prosthesis part having the conical protrusion can preferably be a prosthesis shaft, and the prosthesis part having the conical bore is preferably a prosthetic joint or part of a prosthetic joint.

The headless screw preferably has a tapered tip which can be engaged with the recess on the cone attachment such that pre-tensioning is generated in the conical connection. According to the principle of the inclined plane, if there is displacement along the axis of the headless screw (generally by turning the headless screw) and interaction with the recess in the cone attachment, the tapered tip can generate pre-tensioning in the conical connection which prevents relative movement of the two prosthesis parts. The tapering tip can thereby interact with an edge of the recess in the cone attachment. However, it is also possible for the recess to have a sliding surface that is complementary to the tapering tip of the headless screw.

Furthermore, the invention relates to a set comprising a first prosthesis part according to the invention having a conical protrusion, and a second and a third prosthesis part, each having a complementary conical bore. Therefore, the first prosthesis part can establish a conical connection both with the second and with the third part.

The second prosthesis part has a through-hole through which a securing element is passed so that when the first prosthesis part is inserted, the securing element can engage in the lateral recess of the cone attachment. The conical bore in the second prosthesis part can be made with a thin or with a thick wall.

The third prosthesis part has a through-hole through which a securing element is passed so that when the first prosthesis part is inserted, the securing element can engage in the lateral recess of the conical protrusion. A cavity is provided in the third prosthesis part for receiving the cone attachment on the first prosthesis part. However, it is also possible for there not to be a corresponding cavity in the third prosthesis part. In this case the cone attachment must then be removed from the first prosthesis part so that the first and the third prosthesis part can be connected.

Independently of how the third prosthesis part is formed, the first prosthesis part can be connected optionally to the second or the third prosthesis part and be secured by a securing element. Therefore, the first prosthesis part is extremely flexible in its use because it can be connected to different configurations of prosthesis parts with a conical bore.

Furthermore, the set can also have at least one securing element, the securing element preferably being a headless screw. It is also preferred if the through-holes in the second and the third prosthesis part have at least partially an internal thread with the same diameter so that a securing element can optionally be used with the second or the third prosthesis part.

The headless screw preferably has a tapering tip which can be engaged with the recess on the cone attachment or on the conical protrusion such that pre-tensioning is generated in the cone connection. According to the principle of the inclined plane, if there is displacement along the axis of the headless screw (generally by turning the headless screw) and interaction with the recess in the cone attachment or on the conical protrusion, the tapering tip can generate pre-tensioning in the conical connection which prevents relative movement of the two prosthesis parts. The tapering tip can interact here with an edge of the recess in the cone attachment or on the conical protrusion. However, it is also possible for the respective recess to have a sliding surface complementary to the tapering tip.

For the person skilled in the art it is obvious that the individual aspects of the invention which were explained in connection with the prosthesis part, the prosthesis or the set of prosthesis parts, can also be applied to the respective other subject matter of the invention.

In the following the invention is described using examples with reference to the attached drawings. These show as follows:

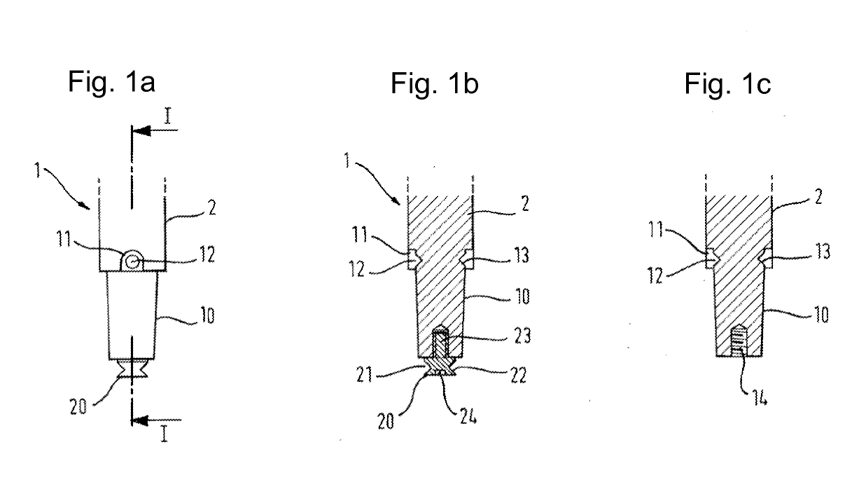

FIGS. 1a-c: different views of an exemplary embodiment of a prosthesis part according to the invention;

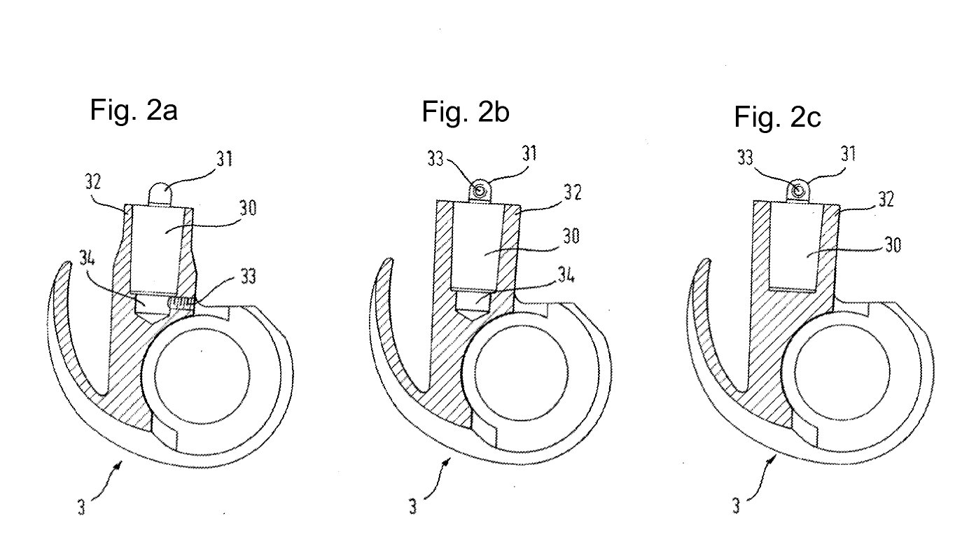

FIGS. 2a-c: three exemplary embodiments of additional prosthesis parts to which the prosthesis part from FIG. 1 can be connected;

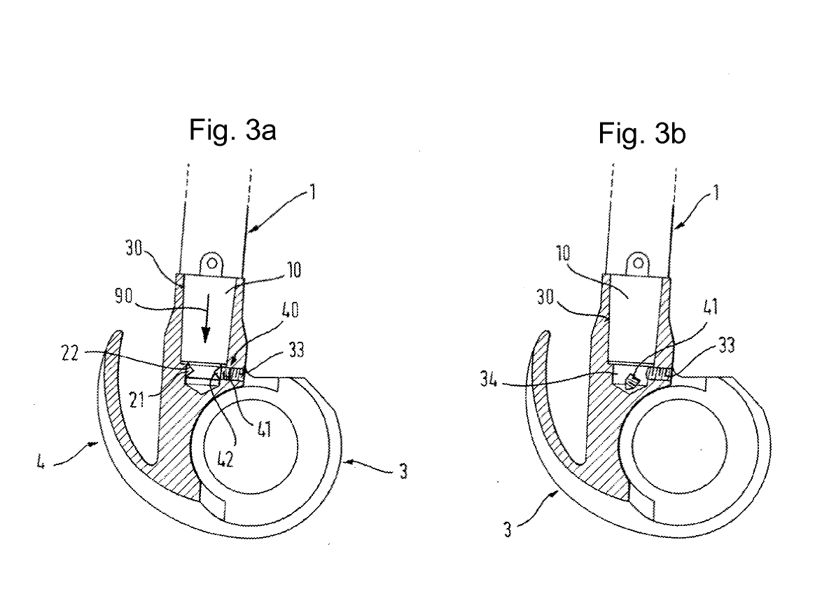

FIGS. 3a, b: an exemplary embodiment of a prosthesis according to the invention; and

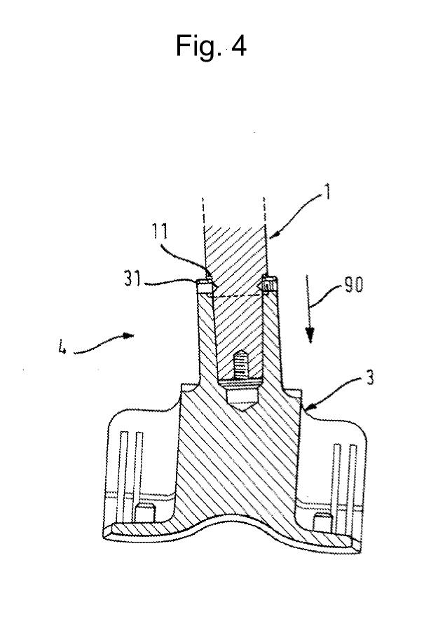

FIG. 4: an additional exemplary embodiment of a prosthesis according to the invention; and

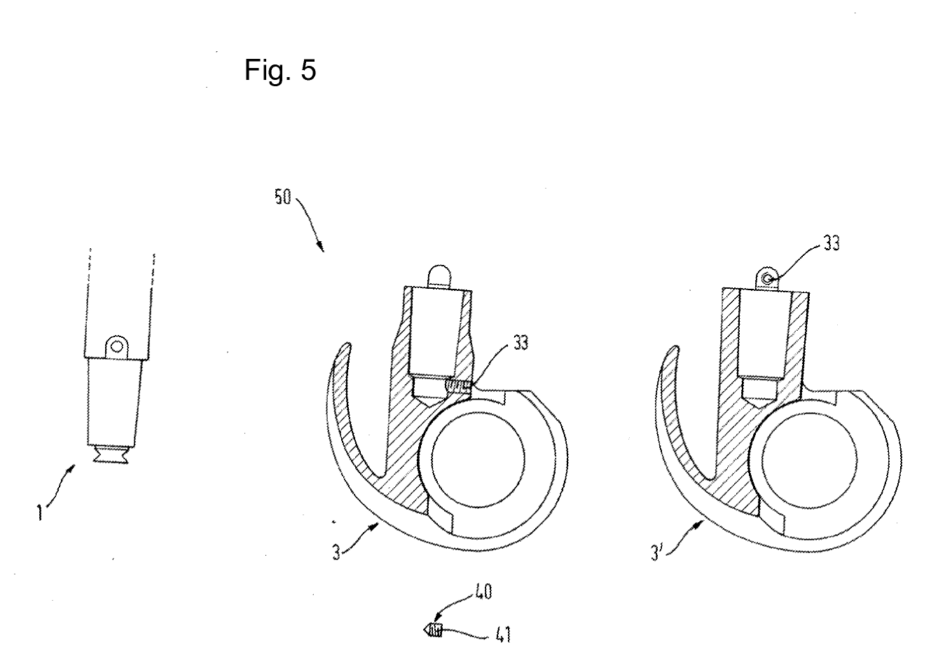

FIG. 5: an exemplary embodiment of a set of prosthesis parts according to the invention.

FIGS. 1a-c show a prosthesis part 1 according to the invention. FIG. 1a shows here a side view, while FIGS. 1b and 1c show sectional illustrations along section line I-I in FIG. 1a.

The prosthesis part 1 according to the invention, which in the exemplary embodiment shown is a prosthesis shaft 2 which is only partially illustrated, has a conical protrusion 10. With this conical protrusion 10 it is possible to connect the prosthesis part 1 to an additional prosthesis part 3 (see FIG. 2). A corresponding conical connection is shown, for example, in FIG. 3, and will be explained further in this regard.

Furthermore, the prosthesis part 1 has two indentations 11 in which, for example, positioning tongues 31 of an additional prosthesis part 3 (see FIG. 2) can engage so as to thus prevent rotation of the conical connection.

At the bottom of these indentations a recess 12 is respectively provided in which a securing element 40 (see FIG. 4) can engage. These recesses 12 are in the form of a blind hole with a tapering bottom 13. However, it is of course also possible for the bottom 13 to be level and/or inclined.

The recesses 12 in the exemplary embodiment shown are clearly assigned to the conical protrusion 10, even if they are not arranged over the conical surface of the protrusion 10. That is to say, the conical protrusion according to the invention does not just include the regions which are actually conical in form, but also the elements and features which are elementary to the formation and securing of a conical connection.

Furthermore, a cone attachment 20 is provided on the conical protrusion 10. This cone attachment 20 can be made to be removable and can be rotationally symmetrical in form. If the cone attachment 20 is fastened to the conical protrusion 10, its axis of symmetry coincides with the axis of the conical protrusion 10 (see FIGS. 1a and 1b).

The cone attachment 20 has a circumferential groove 21, the bottom 22 of which tapers. However, it is also possible for the groove bottom to be made level and/or inclined. Moreover, the cone attachment 20 also has a screw-like extension 23 with which it can be fastened to the conical protrusion 10 by a threaded bore 14. In order to facilitate the removal of the cone attachment 20, which may be necessary, a moulded indentation 24 is also provided for the engagement of an Allen key. By using an appropriate tool the cone attachment 20 can be removed easily from the prosthesis part 1 or the conical protrusion 10. In FIG. 1 c the prosthesis part 1 is shown with the cone attachment 20 removed.

In FIGS. 2a-c three additional prosthesis parts 3 are shown as examples, to which the prosthesis part 1 from FIGS. 1 a-c can be connected. In all of the additional prosthesis parts 3 shown, these are femoral components of a knee joint prosthesis.

All of the prosthesis parts 3 from FIGS. 2a-c have a conical bore 30, and these are configured complementarily to the conical protrusion 10 of the prosthesis part 1 from FIGS. 1a-c. Moreover, they have tongues 31 which can interact with the indentations 11 on the conical protrusion 10 of the prosthesis part 1 so as to prevent rotation when the prosthesis parts 1 and 3 are in the assembled state. For technical reasons relating to the illustration, only one tongue 31 is shown in FIGS. 2a-c.

In the prosthesis part 3 according to FIG. 2a, the conical bore 30 is made with a thin wall, i.e. the wall 32 surrounding the conical bore 30 has a wall thickness that is too small to be able to ensure to a sufficient extent that a through-hole 33, through which a securing element (not shown) is passed, will not shear. In fact, a corresponding opening 33 is disposed in a region away from the conical bore 30 where there is sufficient wall thickness.

The through hole 33, which has an internal thread, ends in a cavity 34 on the bottom of the conical bore 30. The cavity 34 is designed to hold the cone attachment 20 on the conical protrusion 10 of the prosthesis part 1 from FIGS. 1a and 1b. A securing element that is passed through the through-hole 33 can interact with the cone attachment 20 in order to secure the conical connection between the prosthesis part 3 from FIG. 2a and a prosthesis part according to FIGS. 1a and 1b. This will be explained in more detail below. As shown, the cavity 34 can be of a particular shape. However, it is also possible for the cavity 34 to be configured as an extension of the conical bore 30.

In FIG. 2b an additional prosthesis part 3 with a conical bore 30 is shown. Unlike the prosthesis part 3 from FIG. 2a, the conical bore 30 here is however made with a thick wall. The wall 32 surrounding the conical bore 30 is therefore sufficiently thick in order to be able to provide a through-hole 33 for passing through a securing element (not shown) without there being any risk of this through-hole 33 shearing.

In the prosthesis part 3 according to FIG. 2b, two through-holes 33 are provided which are respectively provided in the region of a tongue 31 and which have a thread corresponding to the internal thread of the through-hole 33 of the prosthesis part 3 from FIG. 2a. For technical reasons relating to the illustration, only one tongue 31 and one through-hole 33 is shown in FIG. 2b.

Accordingly, the through-holes 33 are arranged such that a securing element (not shown) guided through one of the through-holes 33 can engage in one of the recesses 12 on the conical protrusion 10 of the prosthesis part 1 from FIGS. 1a-c in order to secure a corresponding cone connection.

The prosthesis part 3 according to FIG. 2b has a cavity 34 which is designed for possibly receiving the cone attachment 20 of the prosthesis part 1 from FIGS. 1a and 1b. Even if the cone attachment 20 is not required to secure the conical connection between the prosthesis parts according to FIGS. 1 a, b and 2b in a prosthesis part 3 according to FIG. 2b, the cone attachment 20 must not necessarily be removed from the conical protrusion 10 of the prosthesis part 1 according to FIGS. 1a and 1b.

The prosthesis part 3 according to FIG. 2c corresponds to a large extent to that of FIG. 2b, and this is why reference is made to the comments made in this regard. However, in the prosthesis part 3 according to FIG. 3c there is no cavity 34 (see FIG. 2b) for receiving the cone attachment 20. Before the prosthesis part 1 according to FIGS. 1a-c can be connected to the prosthesis part 3 from FIG. 2c, the cone attachment 20 must first of all be removed from the prosthesis part 1 (see FIG. 1 c).

FIG. 3a shows how the prosthesis parts 1 and 3 from FIGS. 1a, b and 2a can be assembled to form a prosthesis 4 according to the invention. The conical protrusion 20 of the one prosthesis part 1 is introduced here into the conical bore 30 of the additional prosthesis part 3. This is therefore a classical conical connection between the two prosthesis parts 1, 3. The tongues 31 on the additional prosthesis part 3 engage here in the indentations 11 of the prosthesis part 1 so that rotation is prevented.

A securing element 40 is guided through the through-hole 33. The securing element 40 is a headless screw 41 with a tapering tip 42 which engages in the thread of the through-hole 33. On the opposite side of the headless screw 41 there is a moulded indentation (not shown) in which an Allen screw can engage. The moulded indentation on the headless screw 41 can preferably be of the same nominal size as the moulded indentation 24 on the cone attachment 20.

The securing element 40 is passed sufficiently far through the through-hole 33 such that it comes into contact with the cone attachment 20 that is located in the cavity 34. Here the tapering tip 42 of the headless screw 41 interacts with the bottom 22 of the recess such that a pre-tensioning force is induced in the conical connection in the direction of the arrow 90.

In the exemplary embodiment in FIG. 3 the length of the headless screw 41 is chosen such that if the cone attachment 20 is missing, the headless screw 41 falls out of the through-hole 33 into the interior of the prosthesis part with the conical bore, i.e. into the cavity 34 or the conical bore 30. This is shown correspondingly in FIG. 2b. Since the headless screw 41 falls out of the through-hole 33 into the interior of the prosthesis part 3, it is indicated to the operating surgeon that the conical connection between the two prosthesis parts 1, 3 is not secured.

In FIG. 4 it is shown how the prosthesis part 1 from FIG. 1 c can be connected to an additional prosthesis part 3 according to FIG. 2b to form a prosthesis 4 according to the invention.

The conical protrusion 10 of the one prosthesis part 1 is introduced here into the conical bore 30 of the other prosthesis part 3 in order to form a conical connection, the tongues 31 engaging in the indentation 11 so as to prevent rotation.

A headless screw 41 is guided as a securing element through one of the two through-holes 33. Here the headless screw 41 has a tapering tip 42 which interacts with the bottom 13 of the recess 12 on the conical protrusion 10 of the prosthesis part 1 so that a pre-tensioning force is generated in the direction of the arrow 90.

In the exemplary embodiment shown, the prosthesis parts 1, 3 according to FIGS. 1c and 2b are connected to one another. However, it is also possible to connect the prosthesis parts 1, 3 according to FIGS. 1c and 2c or FIGS. 1a, b and 2b to one another in a corresponding manner.

FIG. 5 shows a set 50 according to the invention of prosthesis parts 1, 3, 3′. The first prosthesis part corresponds here to that of FIG. 1, while the second and the third prosthesis part 3, 3′ correspond to those of FIGS. 2a and 2b. The set 50 of prosthesis parts 1, 3, 3′ further comprises a headless screw 41 as a securing element 40. Provision is made such that the through-holes 33 of the prosthesis parts 3, 3′ have identical internal threads so that the headless screw 41 can be used both with the one prosthesis part 3 and with the other prosthesis part 3′. With regard to the possibilities for interconnecting the individual prosthesis parts 1, 3, 3′, reference is made to the comments made above.

1. A prosthesis part comprising:
a conical protrusion for connecting said prosthesis part to an additional prosthesis part having a complementary conical bore, the conical protrusion including a lateral recess configured to engage a first securing element, wherein
said prosthesis part having the conical protrusion includes a removable cone attachment having a lateral recess configured to engage a second securing element.

2. The prosthesis part according to claim 1, wherein
the lateral recess for the engagement of a securing element on the conical protrusion of said prosthesis part is in the form of a circumferential indentation or blind hole.

3. The prosthesis part according to claim 1, wherein
the removable cone attachment is rotationally symmetrical and generally cylindrical in form.

4. The prosthesis part according to claim 3, wherein
an axis of symmetry of the removable cone attachment in an assembled state of said prosthesis part coincides with an axis of the conical protrusion.

5. The prosthesis part according to claim 1, wherein
the second lateral recess on the removable cone attachment is in the form of a circumferential indentation or blind hole.

6. The prosthesis part according to claim 1, wherein
the removable cone attachment is connected to the conical protrusion such as to be removable by means of a screw connection,
the removable cone attachment having an extension in the form of a screw, and
the conical protrusion having a blind hole with an internal thread in order to form the screw connection.

7. A prosthesis comprising said prosthesis part according to claim 1 having the conical protrusion, the additional prosthesis part having the complementary conical bore and the first securing element, the additional prosthesis part having the conical bore having a through-hole through which the first securing element is passed so that the second securing element can be engaged with the lateral recess of the removable cone attachment of said prosthesis part having the conical protrusion that is inserted into the additional prosthesis part having the conical bore, wherein
the securing element is a headless screw, the length of which is chosen such that, if the removable cone attachment or the prosthesis part having the conical protrusion is missing, it can fall out of the through-hole in order to pass the securing element into the interior of the prosthesis part having the conical bore.

8. The prosthesis according to claim 7, wherein
said prosthesis part having the conical protrusion is a prosthesis shaft and/or
the additional prosthesis part having the conical bore is a prosthetic joint or part of a prosthetic joint.

9. The prosthesis according to claim 7, wherein
the headless screw comprises a tapered tip configured to engage with the second lateral recess on the cone attachment such that pre-tensioning is generated in the conical connection.

10. A set of prosthesis parts comprising:
a first prosthesis part according to claim 1 having the conical protrusion;
a second prosthesis part and a third prosthesis part, each having a complementary conical bore, wherein
the second prosthesis part includes a through-hole through which a securing element is passed so that the securing element can be engaged with the lateral recess of the removable cone attachment of the first prosthesis part inserted into the second prosthesis part, and
the third prosthesis part includes a through-hole through which a securing element is passed so that the securing element can be engaged with the lateral recess of the conical protrusion of the first prosthesis part inserted into the third prosthesis part.

11. The set according to claim 10, wherein
the headless screw comprises a tapered tip which can optionally be engaged with the lateral recess on the cone attachment or on the lateral recess on the conical protrusion such that pre-tensioning is generated in the conical connection.

12. The prosthesis part according to claim 2, wherein
the removable cone attachment is rotationally symmetrical and generally cylindrical in form.

13. The prosthesis according to claim 8, wherein
the headless screw comprises a tapered tip configured to engage with the second lateral recess on the cone attachment such that pre-tensioning is generated in the conical connection.
